(19) United States
(12) Patent Application Publication
(10) Pub. No.: US 2026/0083301 A1
(43) Pub. Date: Mar. 26, 2026

(54) STEERABLE ULTRASOUND ATTACHMENT FOR ENDOSCOPE

(71) Applicant: EndoSound, Inc., Portland, OR (US)

(72) Inventors: Stephen Edward STEINBERG, Boca Raton, FL (US); Scott Sutherland CORBETT, III, Portland, OR (US)

(73) Assignee: EndoSound, Inc., Portland, OR (US)

(21) Appl. No.: 19/407,765

(22) Filed: Dec. 3, 2025

Related U.S. Application Data
(63) Continuation-in-part of application No. 18/188,370, filed on Mar. 22, 2023, now Pat. No. 12,514,432.
Application No. 18/188,370 is a continuation of application No. 17/813,130, filed on Jul. 18, 2022, now abandoned.
Application No. 17/813,130 is a continuation of application No. 17/193,553, filed on Mar. 5, 2021, now Pat. No. 11,419,483.
Application No. 17/193,553 is a continuation of application No. 16/523,971, filed on Jul. 26, 2019, now Pat. No. 11,547,277.
Application No. 16/523,971 is a continuation-in-part of application No. PCT/US2019/027331, filed on Apr. 12, 2019.
Application No. PCT/US2019/027331 is a continuation of application No. 15/951,347, filed on Apr. 12, 2018, now Pat. No. 10,363,014.

(51) Int. Cl. A61B 1/00; A61B 1/005; A61B 1/01; A61B 1/018; A61B 1/05; A61B 1/273; A61B 8/00; A61B 8/12; A61B 10/04; A61B 17/00

(52) U.S. Cl. CPC A61B 1/00101; A61B 1/00097; A61B 1/00098; A61B 1/0014; A61B 1/0051; A61B 1/0057; A61B 1/01; A61B 8/4422; A61B 8/4466; A61B 10/04; A61B 1/00103; A61B 1/00133; A61B 1/00135; A61B 1/018; A61B 1/053; A61B 1/273; A61B 8/12; A61B 8/4411; A61B 8/445; A61B 2010/045; A61B 2017/0034

(57) ABSTRACT

An endoscope add-on assembly adapted to be attached to a target endoscope. The endoscope add-on assembly includes an ultrasound imaging head, an ultrasound imaging sub-assembly, and a needle guide ramp. The ultrasound imaging head defines an imaging slice plane. The ultrasound imaging sub-assembly is configured to selectively engage the ultrasound imaging head. The ultrasound imaging sub-assembly includes a retaining element configured to secure the ultrasound imaging sub-assembly to the target endoscope. The needle guide ramp is configured to direct a motion of a needle of the target endoscope. The needle guide ramp is configured to align the needle of the target endoscope within the imaging slice plane when the endoscope add-on assembly is attached to the target endoscope.

### RELATED APPLICATIONS

This application is a continuation-in-part application claiming priority under 35 U.S.C. § 120 to U.S. patent application Ser. No. 18/188,370, which itself is a continuation of U.S. patent application Ser. No. 17/813,130, entitled STEERABLE ULTRASOUND ATTACHMENT FOR ENDOSCOPE, filed Jul. 18, 2022, which is a continuation application claiming priority under 35 U.S.C. § 120 to U.S. patent application Ser. No. 17/193,553, filed Mar. 5, 2021, which is a continuation application claim priority under 35 U.S.C. § 120 to U.S. patent application Ser. No. 16/523,971, filed Jul. 26, 2019, which itself is a continuation-in-part of PCT International Application PCT/US2019/027331, filed Apr. 12, 2019, which itself is a continuation of application U.S. Ser. No. 15/951,347 filed Apr. 12, 2018, now U.S. Pat. No. 10,363,014, issued Jul. 30, 2019, all of which are incorporated by reference as if fully set forth herein.

### BACKGROUND

### 1. Field of the Invention

The Invention is in the field of ultrasound imaging add-on equipment for endoscopes.

### 2. Background Art

Endoscopic ultrasound has undergone a rapid pace of development, now being used for the diagnosis and treatment of a wide variety of medical problems. As an endoscope can reach a location in the intestinal tract, closer than any skin surface, there is an opportunity to image from a closer location, and to obtain a tissue sample, using a biopsy needle and implement a variety of treatments. But due to an expense of greater than $300,000 for a complete system, endoscopic ultrasound systems are generally restricted to major hospitals. Endoscopes, however, are used in physicians'offices, most outpatient surgery centers and virtually all hospitals.

One type of endoscope is an upper endoscope, used to image and take tissue specimens from the upper GI tract. In this type of endoscope, if a needle is used to collect a specimen, it is typically advanced straight out of an endoscope lumen in a distal direction. Other types of endoscopes are bronchoscopes for viewing air passageways in the lungs and colonoscopes for viewing the colon.

Yet another type of endoscope is a duodenoscope, designed to be introduced into the duodenum (the upper part of the small intestines), and typically used to perform endoscope retrograde cholangiopancreatography (ERCP), in which the duct of the pancreas and liver are imaged. Duodenoscopes are also used to gather tissue biopsies from sites in the duodenum, including the bile ducts. Duodenoscopes typically have a tip that houses a light, a video camera, and an instrument guide that can be tilted by an operator to control the angle at which the instrument (needle or other type of instrument) advances. Although the video camera and light can produce imagery that may help the endoscopist visualize potential targets directly, ultrasound imagery, when available, provides a different, dramatically expanded view of the regional anatomy. An ultrasound add-on for endoscopes has been described, but its capabilities are limited in that the viewing angle of the imaging head cannot be adjusted, and it does not provide for tissue sampling.

A problem faced by practitioners in the field of endoscopy is the thorough disinfection of the endoscope, between uses. As many endoscopes, in particular duodenoscopes, have some mechanical complexity, introducing a sterilizing material into the small spaces defined by these mechanisms, creates a huge challenge.

Recently, an endoscope mechanism, having an instrument angle adjustment mechanism that is removable and disposable has been introduced, addressing many of these issues.

### SUMMARY

In a first separate aspect, the present invention may take the form of an endoscope add-on assembly adapted to be attached to a target endoscope. The endoscope add-on assembly includes an ultrasound imaging head, an ultrasound imaging sub-assembly, and a needle guide ramp. The ultrasound imaging head defines an imaging slice plane. The ultrasound imaging sub-assembly is configured to selectively engage the ultrasound imaging head. The ultrasound imaging sub-assembly includes a retaining element configured to secure the ultrasound imaging sub-assembly to the target endoscope. The needle guide ramp is configured to direct a motion of a needle of the target endoscope. The needle guide ramp is configured to align the needle of the target endoscope within the imaging slice plane when the endoscope add-on assembly is attached to the target endoscope.

In a second separate aspect, the present invention may take the form of an endoscope add-on assembly adapted to be attached to a target endoscope. The endoscope add-on assembly includes an ultrasound imaging head and an ultrasound imaging sub-assembly. The ultrasound imaging sub-assembly is configured to selectively engage the ultrasound imaging head. The ultrasound imaging sub-assembly includes a retaining element and an abutment. The retaining element is configured to secure the ultrasound imaging sub-assembly to the target endoscope. The abutment is configured to align the ultrasound imaging sub-assembly in a first predetermined position relative to the target endoscope when secured.

In a third separate aspect, the present invention may take the form of an endoscope add-on assembly adapted to be attached to a target endoscope. The endoscope add-on assembly includes an ultrasound imaging head and an ultrasound imaging sub-assembly. The ultrasound imaging head defines a recess. The ultrasound imaging sub-assembly includes an attachment arm and a retaining element. The attachment arm has a geometry compatible to the recess. The attachment arm is configured to selectively engage the ultrasound imaging head. The retaining element is configured to secure the ultrasound imaging sub-assembly to the target endoscope.

### BRIEF DESCRIPTION OF THE DRAWINGS

Exemplary embodiments are illustrated in referenced drawings. It is intended that the embodiments and figures disclosed herein are to be considered illustrative rather than restrictive.

FIG. 1 is an isometric view of an imaging assembly having an endoscope and an ultrasound imaging assembly added on, according to a preferred embodiment of the present invention.

FIG. 2 is an isometric view of the distal end of the assembly of FIG. 1.

FIG. 3 is a side view of the distal end of the assembly of FIG. 1, in a first position.

FIG. 4 is a side view of the distal end of the assembly of FIG. 1, in a second position.

FIG. 5 is a side view of the distal end of an alternative embodiment of an imaging assembly.

FIG. 6 is a side view of another alternative embodiment of an imaging assembly.

FIG. 7 is an alternative embodiment of an imaging assembly, having an ultrasound imaging sub-assembly that includes an ultrasound head movement assembly that is detachable, and showing three alternative needle guides.

FIG. 8 is a sectional view of a of the assembly of FIG. 7, showing a needle guide in a deployed state.

FIG. 9 is a detail view of a portion of the assembly of FIG. 7, showing two of the parts disassembled from each other.

FIG. 10 is the detail view of FIG. 9 but showing the two parts joined.

FIG. 11 is a sectional view taken along line 11-11, of FIG. 10.

FIG. 12 is a sectional view taken along line 12-12 of FIG. 10.

FIG. 13 is a sectional view of the tip of the assembly of FIG. 7, showing the needle guide used to guide a needle.

FIG. 14 is a sectional partial view of the assembly of FIG. 7 having a different style of needle guide.

FIG. 15 is a view of the assembly of FIG. 14, showing the needle guide in use.

FIG. 16 is an isometric view of a duodenoscope assembly, having an ultrasound imaging sub-assembly.

FIG. 17 is an isometric view of the assembly of FIG. 16, in a disassembled state.

FIG. 18 is a sectional view of the assembly of FIG. 16, showing a different position for a portion of the assembly, in dashed line.

FIG. 19 is an isometric view of an alternative embodiment of a duodenoscope assembly.

FIG. 20 is an isometric of the assembly of FIG. 19, in a disassembled state.

FIG. 21A is an isometric view of a needle guide ramp.

FIG. 21B is a side view of the needle guide ramp of FIG. 21A.

FIGS. 22A and 22B are views of an imaging assembly having an endoscope and an ultrasound imaging assembly comprising a needle guide ramp, according to a non-limiting embodiment of the present disclosure.

FIG. 23 is a side view of an imaging assembly having an endoscope and an ultrasound imaging assembly comprising a needle guide ramp and a tension member, according to a non-limiting embodiment of the present disclosure.

FIGS. 24A and 24B are views of an imaging assembly having an endoscope and an ultrasound imaging assembly comprising an attachment arm, according to a non-limiting embodiment of the present disclosure.

FIG. 25 is an isometric view of an imaging assembly having an endoscope and an ultrasound imaging assembly comprising an abutment, according to a non-limiting embodiment of the present disclosure.

### DETAILED DESCRIPTION

As used in this application, the term “endoscope” refers to an illuminated optical, typically a slender and tubular instrument used to look deep into the body and used in procedures referred to as “endoscopy”. This term encompasses, but is not limited to upper endoscopes, duodenoscopes, colonoscopes and bronchoscopes, as well as devices referenced simply as “endoscopes”.

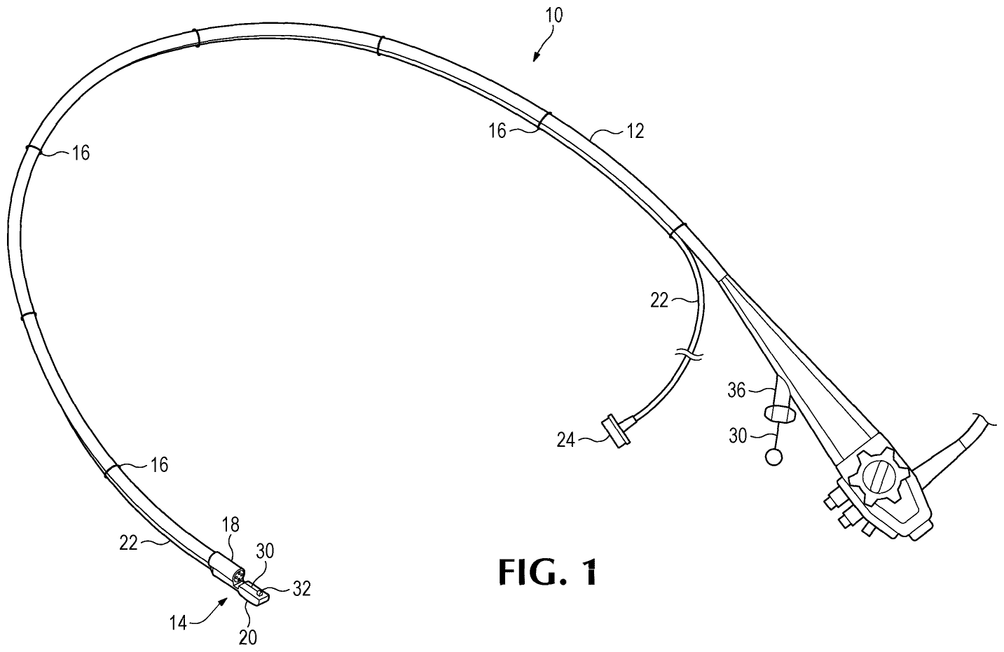

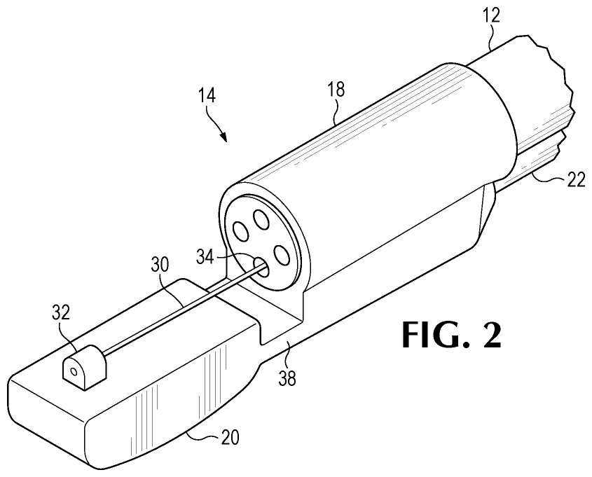

In a first preferred embodiment, an imaging assembly 10 includes an upper endoscope 12 and an ultrasound assembly 14 that has been attached to endoscope 12 by means of retaining element 18, integral to ultrasound assembly 14. In an alternative preferred embodiment, a retaining element is provided that is separate from ultrasound assembly 14 but works cooperatively to retain assembly 14 on endoscope 12. Assembly 14 also includes an ultrasound imaging (also referred to as “transducer”) head 20 that is electrically connected to a multiple signal pathway cable 22 by way of a flex circuit 50 (which is also a form of a signal pathway cable), that includes a set of parallel electrical leads, which may be traces. Cable 22, which has a multiplicity of signal pathways extending therethrough terminates in a connector 24, adapted to connect to an imaging station. Elements 16, which may be rubber bands, or some other form of elastic bands or clips, help to retain cable 22, to the side of endoscope 12. A tension member 30, such as a wire (which may also have some compressive strength) is attached to a fixation point 32 on ultrasound imaging head 20 and extends through a lumen 34 (FIG. 2) to emerge outside of a port 36 on the proximal end of endoscope 12, to be manipulated. In embodiments, tension member 30 does not extend through lumen 34, but extends along the side of endoscope 12, and in some embodiments is retained by elements 16, which are modified from the simple shapes shown in FIG. 1, to include eyelets, to create a guide path for tension member 30. In one embodiment, tension member 30 is connected to controls on the proximal end of endoscope 12, to facilitate manipulation. In embodiments, these controls may take the form of a spool, that can be easily let out, or drawn in. Endoscope 12 also is equipped with intrinsic controls for deflecting the tip of the insertion tube, to facilitate introduction to a site of interest.

In an alternative embodiment, tension member 30 is replaced by a tension member extending along the exterior of the endoscope, to a fixation point on the end of the endoscope. A physician may exert traction or pulsion on tension member 30 in any one of a variety of ways, to cause ultrasound imaging head 20 to bend forward or back toward retaining element 18, as permitted by a resiliently flexible neck 38 (FIG. 2). In one method a rotatable element is used to draw in tension member 30 or push it out.

In preferred embodiment endoscope 12 includes an element at its distal end to guide the alignment of the retaining element 18. For example, endoscope 12 many include a groove at its distal end, into which a key element on retaining element 18 engages. In another embodiment, an orientation guide includes a peg that fits into the lumen 34 and is used to guide the correct orientation of retaining element 18. In one embodiment, assembly 14 is made for intended disposal, after a single use, and is used in this manner. In another embodiment, assembly 14 is constructed so as to be prepared and/or cleaned appropriately for reuse, after use, and then reused. Although until recently, generally, disinfection procedures were deemed adequate, the detection of instances of the spread of infection through endoscope has given rise to the use of high-end disinfection techniques for endoscopes. These disinfection techniques make use of chemicals to kill any pathogens left on the scope after use. Other disinfection or sterilization techniques may be used, including using of UV light and/or a gas, such as o-zone. In the context of this application, the term “cleaning” encompasses all disinfection and sterilization techniques. Generally, the materials used in endoscopes are such that autoclaving an endoscope, or an attachment thereto is not feasible.

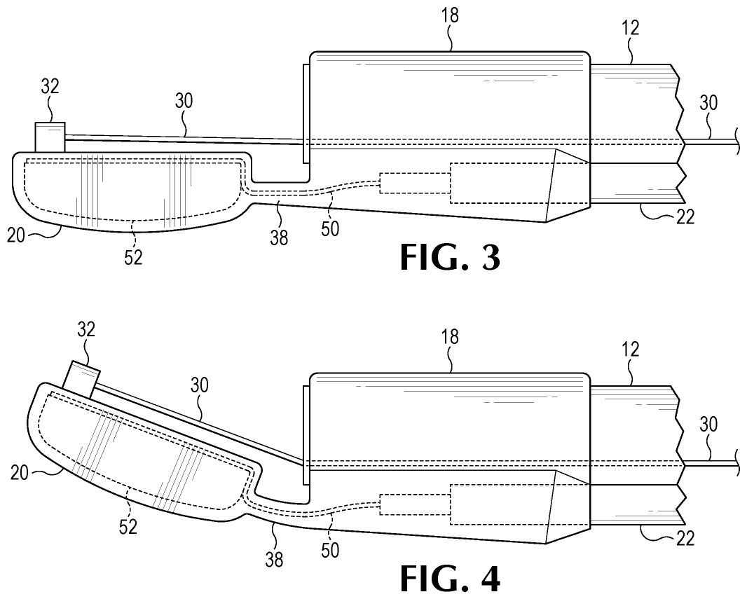

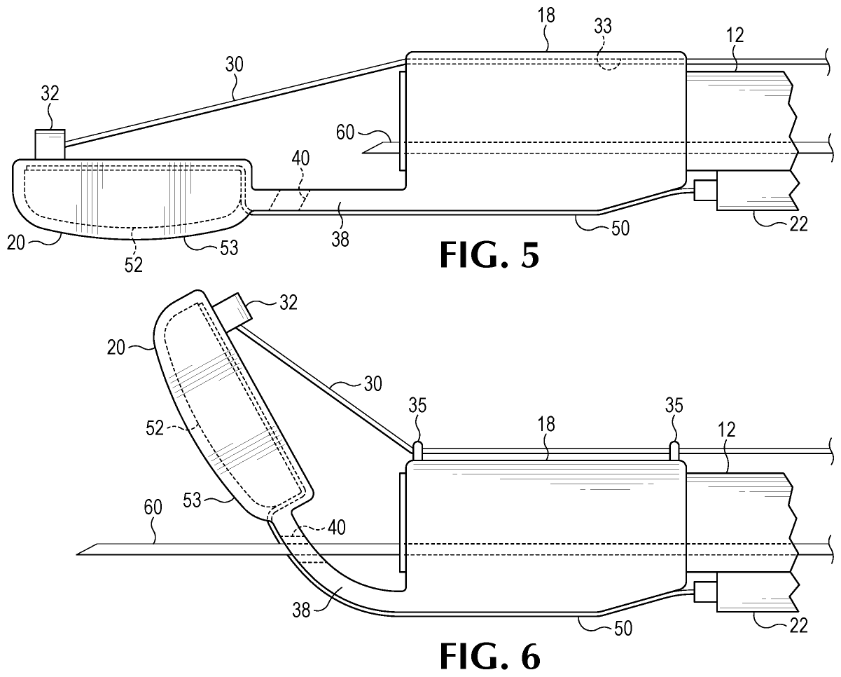

Referring to FIGS. 3 and 4, a flex circuit 50, which passes through the flexible neck 38, electrically connects imaging head 20 to cable 22. Flex circuit 50 has an electrical lead for each transducer element in an ultrasound element array 52, resident in the ultrasound imaging head 20, to drive ultrasound element array 52 and relay signals from it. Array 52 is covered with a protective coating 53 (FIGS. 5 and 6). In an alternative preferred embodiment, flex circuit 50 extends from imaging head 20 to (or through) connector 24, may define a plurality of coax cables and may directly contact the elements of the ultrasound array 52. In another alternative embodiment, cable 22 comprises a set of coax cable bound together with an adherent and protective substance, such as a polymer, and extends from connector 24 to imaging head 20. In yet another embodiment, a fiber optic cable is used in place of cable 22, with light to electric convertors at its distal end. In any one of these arrangements elements 22 and 50 could be termed separately or in combination as a multiple signal pathway cable.

In a preferred embodiment, a biopsy needle 60 (FIGS. 5 and 6), which forms the sharpened, distal portion of a long, flexible, hollow-core wire, is provided. This wire is sheathed in a flexible conduit (not shown), thin enough to extend through the lumen 34 and protecting endoscope 12 from being damaged by needle 60. Once the conduit reaches the distal end of endoscope 12, it may be pushed out to extend from lumen 34, and provide further guidance for needle 60, which is pushed out of the conduit at a point distal to the end of endoscope 12. Alternatively, the conduit may be pushed roughly to the end of lumen 34, with the needle 60 pushed out of the conduit at that point.

Referring to FIG. 5, in an alternative preferred embodiment tension member 30 extends through a channel 33 in retaining element 18 to reach fixation point 32. This figure also shows a needle 60 that has been pushed through a lumen of the endoscope 12 and is emerging from the distal end of the lumen. An aperture 40 is defined in neck 38, corresponding to an aperture in flex circuit 50, aligned with aperture 40. FIG. 6 shows an embodiment that is similar to that of FIG. 5, but instead with tension member 30 extending through a pair of eyelets 35, supported on the retaining element 18. As well as showing a slightly different embodiment, FIG. 6 also shows imaging head 20 retracted and needle 60 extending through aperture 40, as it would be in order to take a biopsy. Notably, in this position the needle would be within the field of view of ultrasound array 52. In embodiments, tension member 30 can pull head 20 into an obtuse angle, relative to the distal end of the endoscope 12. Generally, aperture 40 is in the shape of a long oval, so that the needle 60 can pass through it over a long range of degree of bending of neck 38. In another preferred embodiment, the flexible conduit is extended distally from lumen 34 into av-shaped indentation (not shown) on surface of flexible neck 38, aligning the conduit so that the needle 60 is aligned to pass through aperture 40.

To use imaging assembly 10, ultrasound assembly 14 is attached to endoscope 12 by means of retaining element 18. In an alternative embodiment, rubber bands or clips 16 (FIG. 1) retain cable 22 to the side of endoscope 12. Imaging head 20 is then delivered to an area of interest, by means of standard endoscope introduction techniques. Imaging head 20 may then be moved to gain imagery of the area of interest by dedicated controls which control the ultrasound imaging head 20 deflection. If there appears to be a finding to be sampled, needle 60 may be introduced through an endoscope lumen and through aperture 40 and used to take a biopsy, inject a drug, or otherwise effect a medical procedure. Finally, needle 60 is retracted through the lumen of endoscope 12 and the endoscope is retrieved from the patient's body. In other embodiments, needle 60 is not included and an assembly that is similar to imaging assembly 10 but without needle 60 and related elements, is used for imaging alone, or for introduction of some other device.

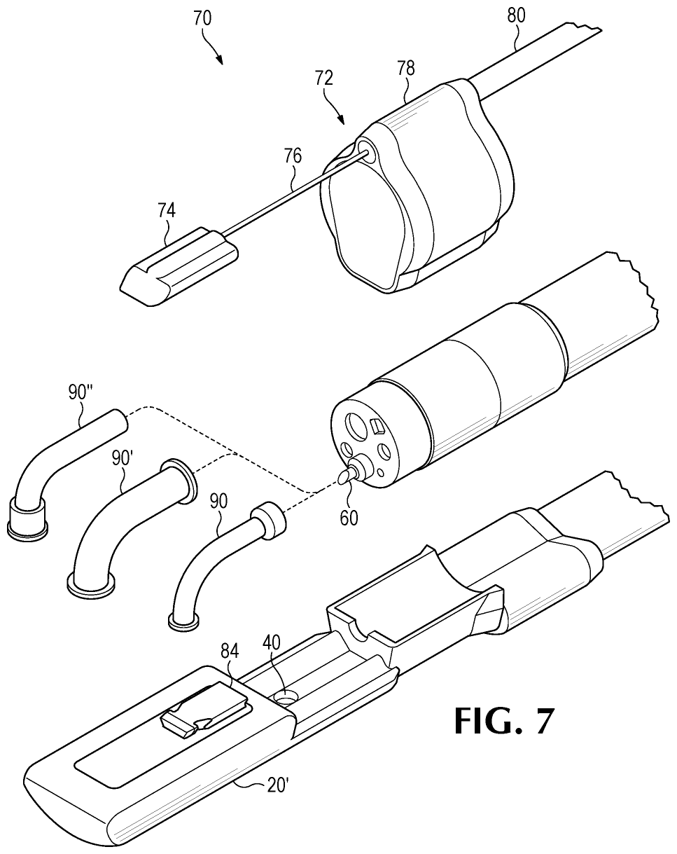

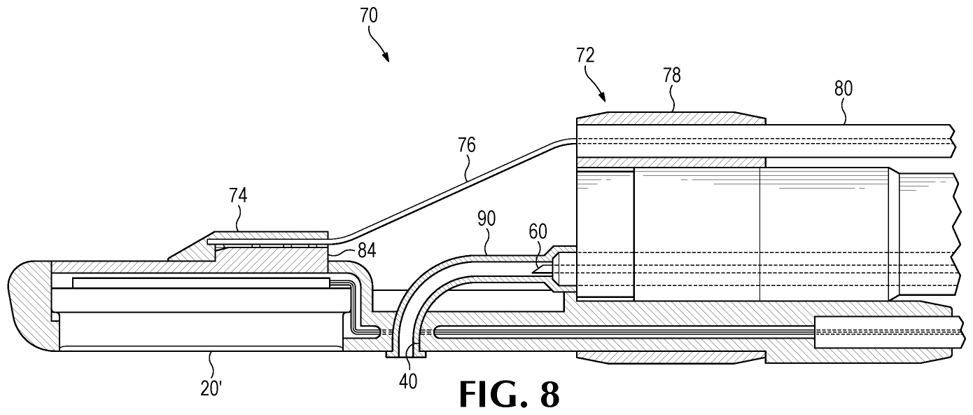

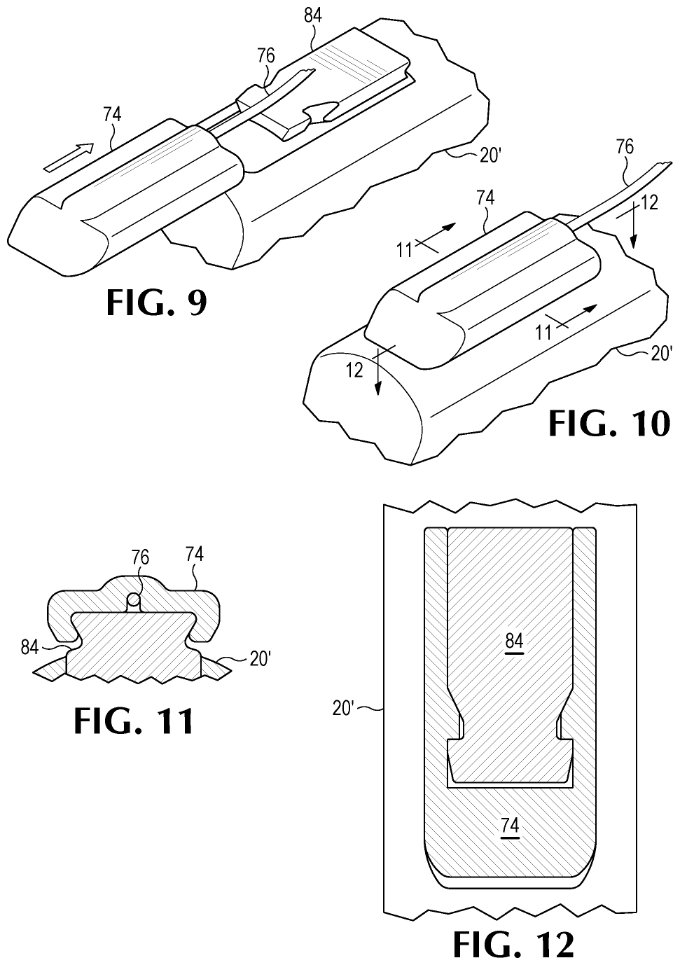

FIGS. 7-13 show an alternative embodiment 70 of the assembly 10, with the further innovation of a disposable head-movement sub-assembly 72, which includes a head clip 74, a movement cable 76, a cable clip 78 and a conduit 80, holding the major portion of movement cable 76. A clip-hold 84 is defined on the back of imaging head 20′. Further, the cable clip 78 holds imaging head and other portions of the ultrasound assembly 14′, including communicative cable 22′, to the endoscope 12. FIGS. 9-12 show engagement of head clip 74 to clip-hold 84. FIG. 9 shows head clip 74 distal to and being pulled back onto clip-hold 84, with FIG. 10 showing head clip 74 engaged to clip-hold 84 and FIGS. 11 and 12 showing different sectional views of head clip 74 and clip-hold 84 engaged together.

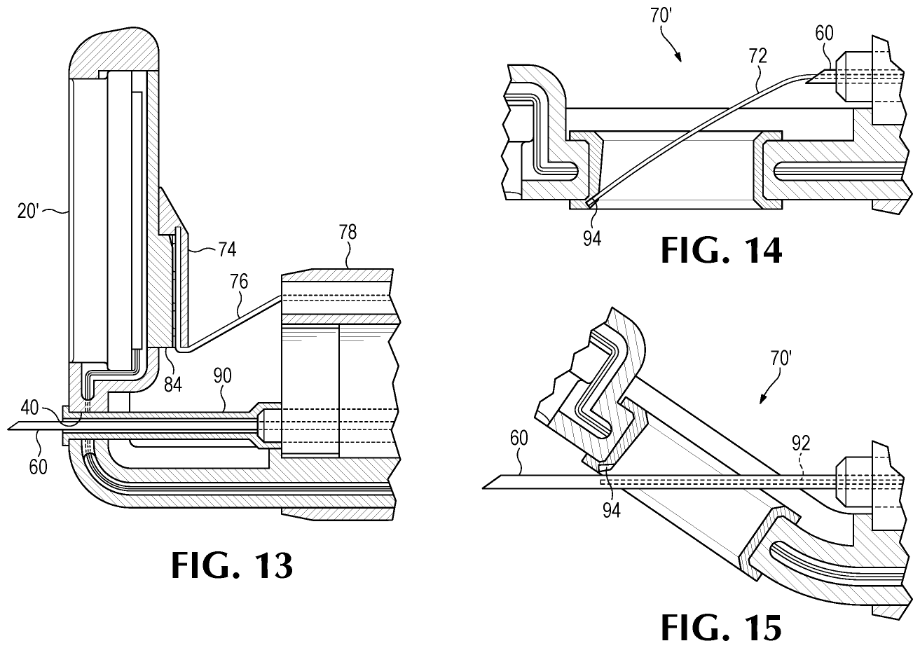

Another difference between assembly 70 and assembly 10 is the optional presence of a needle guide 90. FIG. 7 shows two additional variant needle guides 90′and 90″. In assembly 10 it is possible that a needle 60 pushed out of a lumen of endoscope 12 could miss the aperture 40 in neck 38 and be blocked by neck 38 from further advancement. This might happen if a user attempted to push needle 60 into use when the neck 38 was not sufficiently pulled back, to bring aperture 40 into the correct position to let needle 60 pass through. The result could be damage caused to imaging head 20′, cause by needle 60. A needle guide 90 engages with aperture 40, so that needle 60 will be guided to aperture 40 with certainty, or will be blocked by guide 90, when head 20′is not positioned correctly to align aperture 40 with the path of needle 60. FIG. 13 shows a needle guide 90 in use as head 20′is pulled fully back, to a forward-looking position as needle 60 is advanced through aperture 40, with the assistance of guide 90. It is a further advantage of assembly 70 (and assembly 10) that the head 20′can be moved to a forward-looking position as shown in FIG. 13, which is helpful to surgeons for some types of procedures. Referring to FIGS. 14 and 15, in a variant 70′to assembly 70, a needle guide 92 is provided in the form of a wire that needle 60 advances over. When not in use, needle guide 92 is retained in a needle-guide notch 94 (FIG. 15).

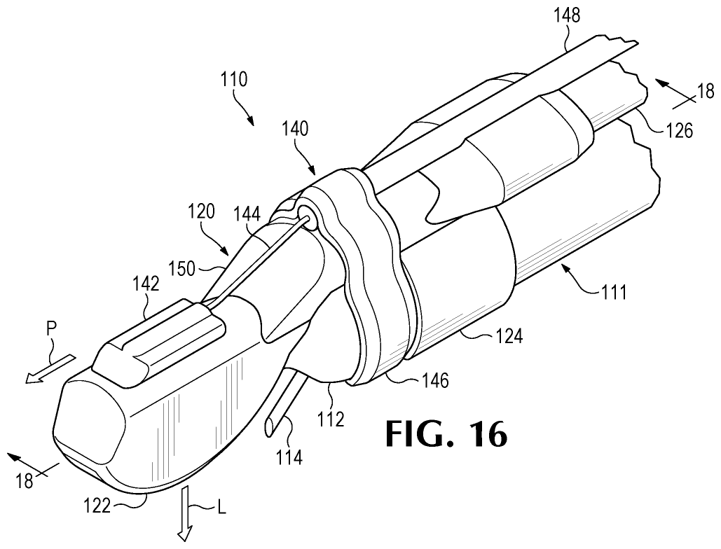

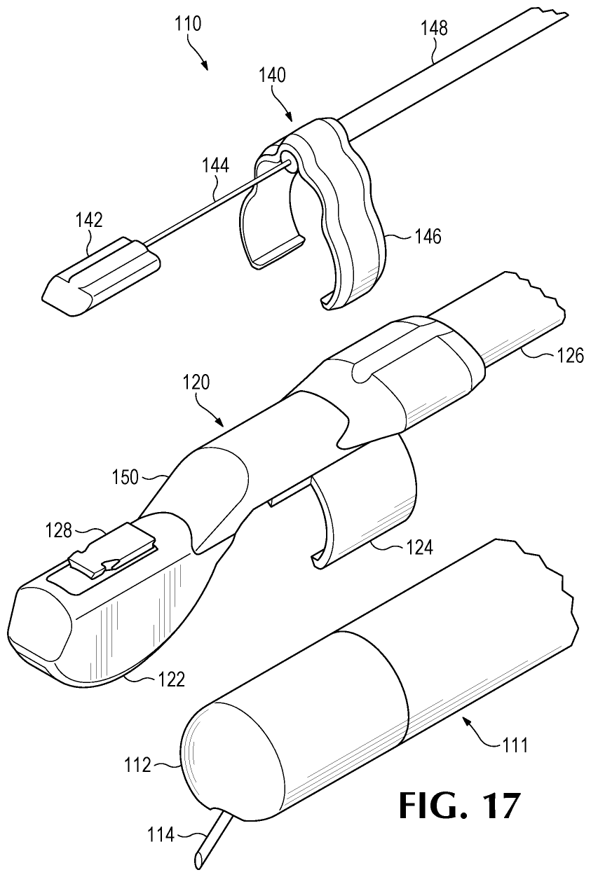

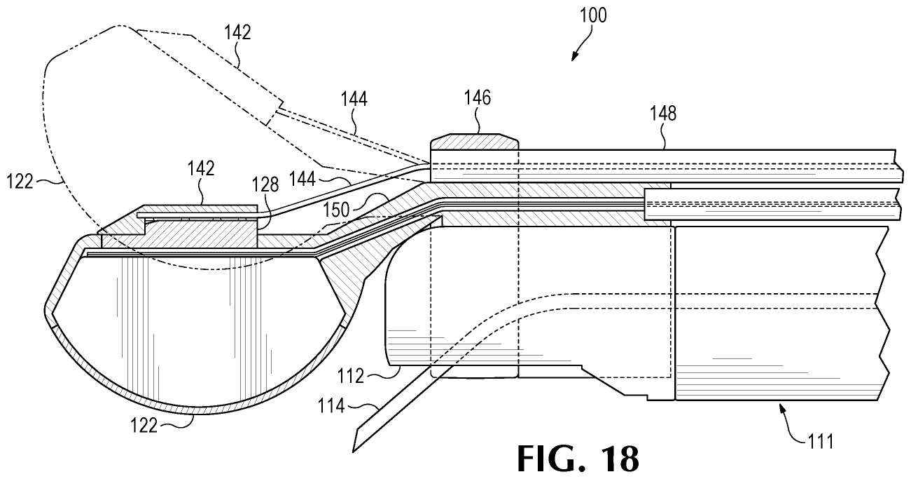

Referring now to FIGS. 16-18, a duodenoscope assembly 110 includes a duodenoscope 111 having a single-use instrument guidance head 112 (shown most clearly in FIG. 18), having an instrument guide 114 extending outwardly at an angle between a first lateral direction Land the distal direction P. Guidance head 112 can change the direction of guide 114, in response to varying user input via a tension member and an instrument variable guide member (not shown). Referring to FIG. 16, a cable/head sub-assembly 120 includes an ultrasound imaging head 122, a scope clip 124, a multiple signal pathway cable 126, delivering signals to imaging head 122 and relaying signals from imaging head 122. The signal pathways of cable 126 may be electrical conductors, and more specifically may each be a coax cable or a trace on a flex circuit. Other forms of signal pathways are possible. Imaging head 122 is shown having a signal emission surface facing the first lateral direction L, and a clip-hold 128 (FIG. 17) is present on head 122 on a side displaced from said signal emission surface in a second lateral direction, opposed to said first lateral direction L. An imaging head movement sub-assembly 140 includes a head clip 142, shaped to engage to clip-hold 128, a movement cable 144, a cable clip 146 and a conduit 148, holding the major portion of movement cable 144. Referring to FIG. 18 when cable 144 is pulled it pulls back imaging head 122 as indicated by the dotted line. In some embodiments sub-assembly 140 further includes an actuator (not shown) at the proximal end, to permit an operator to draw in cable 144, thereby pulling on imaging head 122 or let out cable 144, either pushing on imaging head 122 or permitting the resiliency of the material of cable 126 to place head 122 into a position more aligned with the longitudinal dimension of the duodenoscope 111, at its distal end. The actuator of cable 144 may take the form of a wheel, a lever or any other arrangement convenient to the user.

Because disinfection techniques typically require the application of chemicals in liquid form, thin crevices, into which liquid might not easily flow are generally undesirable. Accordingly, clip-hold 128 is designed so as not to define thin crevices with the imaging head 122. In alternative preferred embodiments, clip-hold 128 may have a shape that is similar to a knob, to further avoid defining any narrow crevices.

As noted in the background, the disinfection of devices such as assembly 110 is a matter of great concern, as there have been cases of the spread of strains of bacteria that are resistant to multiple antibiotics, by way of duodenoscope reuse. One area which may prove particularly difficult to sterilize is conduit 148, as movement cable 144 will tend to introduce body fluids into conduit 148 as cable 144 is pulled back into conduit 148, as imaging head 122 is moved back. To address this issue head movement sub-assembly is releasable and removable from the remainder of assembly 110 and is made to be inexpensive enough to use a single time and then be disposed. This eliminates the possibility of infection being spread from patient to patient by way of sub-assembly 140. Cable/head sub-assembly 120 does not have a similar structure that would provide a hard-to-reach place that would make disinfection difficult and will tend to be more expensive as it must contain a multiplicity of fine wires or other forms of signal pathways. Accordingly, cable/head sub-assembly 120 is designed to be cleaned and reused.

Before performing an endoscopic (duodenoscopic) procedure the endoscopist would obtain an unused head movement sub-assembly 140 and attach it to the remainder of assembly 110. After use, the user detaches and disposes of sub-assembly 140.

Referring to FIG. 16, movement cable 144 may be pulled back to cause head 122 to face in a more distal facing direction. Pushing cable 144 forward causes head 122 to adopt a lateral viewing angle as shown, in one embodiment due to resilience of neck 150, but in another due to stiffness and compressive strength in cable 144.

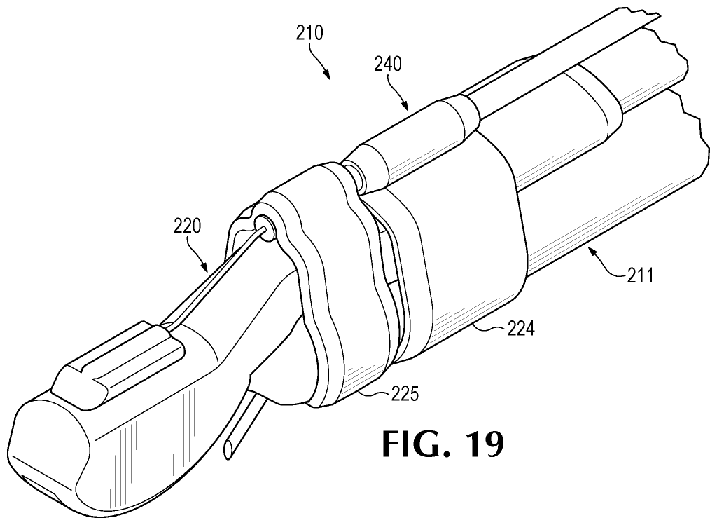

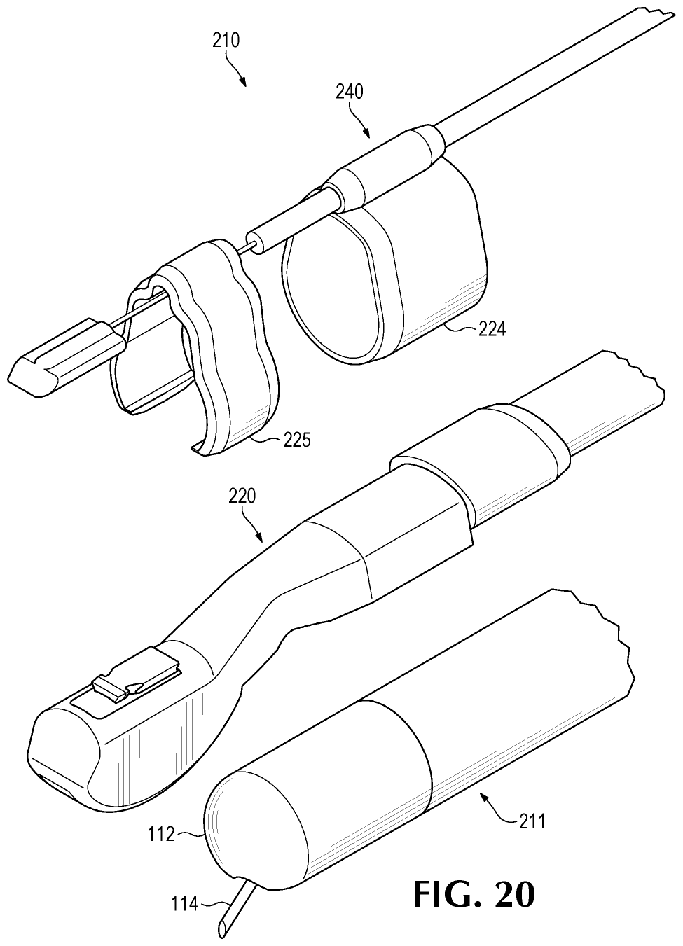

Referring to FIGS. 19 and 20, in an alternative preferred embodiment of a duodenoscope assembly 210, a holder 224 encompasses together both the duodenoscope 211, the cable/head sub-assembly 220 and the imaging head movement sub-assembly 240. A clip 225 also helps to hold the elements together.

In an additional set of embodiments and methods of use, any one of assembly 14 (combined with tension member 30), and assembly 70 and the combination of assemblies 120 and 140, can be made so that the resultant assembly 14/30, 70 or 120/140 (henceforth collective designated as assembly 14′) is produced and sold with a recommended method of use to dispose the entire assembly after a single use. This may greatly simplify health facility operations. In a preferred embodiment, the ultrasound array 52 (or the array in imaging head 20′or 122) is a capacitive micromachined ultrasonic transducer (CMUT), which is generally less expensive than a piezoelectric transducer. Because cleansing an ultrasound assembly 14′can be so cumbersome and expensive, and because of the great value of the surgeries being performed, even an assembly 14′ selling for upwards of $2,000 in 2019, could be more economical to dispose of, than to be cleansed and reused. In one embodiment of an assembly 14′, the number of array elements is reduced, from for example 256, to for example 128, or even to 64, to reduce the cost of the array, and the signal pathways leading to and from the array.

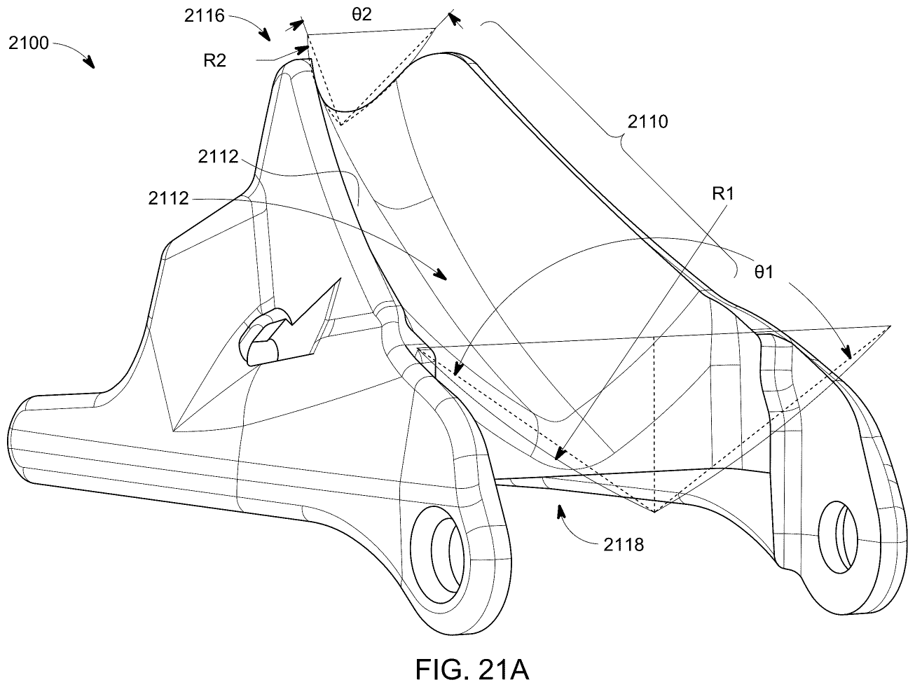

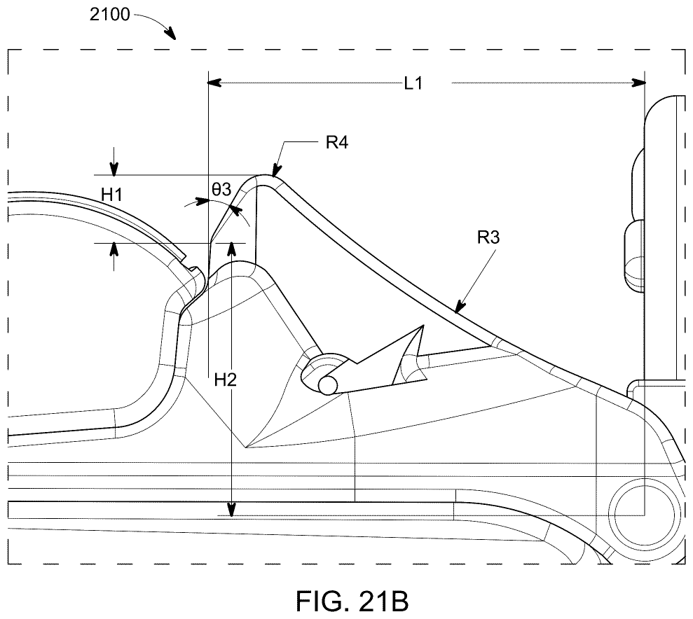

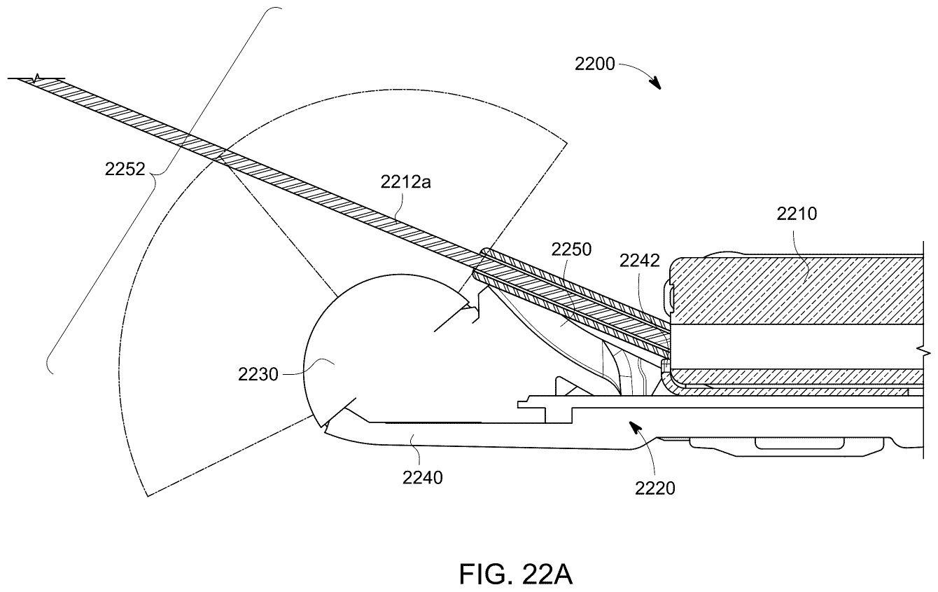

Referring now to FIGS. 21A and 21B, various views of a needle guide ramp 2100 are depicted, according to non-limiting aspects of the present disclosure. FIGS. 21A and 21B depict potential dimensions for the needle guide ramp 2100. The needle guide ramp 2100 may define a v-shaped channel 2110. The v-shaped channel 2110 may be defined by side walls 2112 and a central groove 2118.

The needle guide ramp 2100 is configured to engage with an endoscope to position the needle of an endoscope within a slicing plane of an ultrasound assembly. Ultrasound assemblies are used to obtain sub-topical images of a surface. When used with an endoscope, the ultrasound can provide images of target structures below the surface of tissue. In some instances, the needle of an endoscope is used to penetrate the tissue to reach the target structure. Accordingly, the needle needs to be positioned within view of the ultrasound assembly to reduce risks of improper needle use. The needle guide ramp 2100 provides the v-shaped channel 2110 to properly position the needle relative to the ultrasound head. According to some embodiments, the needle guide ramp may be positioned between the endoscope head and the ultrasound head. According to some examples, the needle guide ramp may be positioned in a range between an eleven to fifteen millimeters between the ultrasound head and the endoscope head. For example, according to one aspect, the needle guide may be positioned between a thirteen-millimeter gap between the ultrasound head and the endoscope head.

The v-shaped channel 2110 provides a route for the needle of an endoscope to reach the slicing plane along the central groove 2114. The needle guide ramp 2100 may be configured to receive the needle at the proximal end 2118 and guide the needle through the distal end 2116 to the slicing plane. The side walls 2112 may provide support to the needle to stay within the central groove 2114.

The side walls 2112 may funnel the needle towards the distal end 2116. According to some embodiments, the side walls 2112 may vary in angle between the proximal end 2118 with an angle θ1 and the distal end 2116 with an angle θ2. According to some embodiments, the proximal angle θ1 of the side walls may be greater than the distal angle θ2 of the side walls to narrow the v-shaped channel at the distal end to ensure the needle is properly aligned by the point it reaches the distal end. According to some examples, the proximal angle θ1 may be greater than ninety degrees while the distal angle θ2 may be less than ninety degrees. According to some examples, the proximal angle θ1 may be greater than one hundred ten degrees while the distal angle θ2 may be less than seventy degrees. In some specific examples, the proximal angle θ1 may be in a range between one hundred ten degrees and one hundred twenty degrees while the distal angle θ2 may be in a range between sixty-five degrees and seventy degrees For example, according to one aspect, the proximal angle θ1 may be one hundred sixteen degrees while the distal angle θ2 may be sixty-eight degrees.

According to some embodiments, the side walls 2112 may following a radius of curvature R1, R2. The side walls 2112 may vary in radius of curvature between the proximal end 2118 with a radius of curvature R1 and the distal end 2116 with a radius of curvature R2. According to some embodiments, the proximal radius of curvature R1 may be greater than the distal radius of curvature R2 to narrow the v-shaped channel at the distal end to ensure the needle is properly aligned by the point it reaches the distal end. According to some examples, The proximal radius of curvature R1 may be in a range between twenty and thirty millimeters, and in some examples, the proximal radius of curvature R1 may be in a range between twenty-three and twenty-five millimeters. According to some examples, The distal radius of curvature R2 may be in a range between three and seven millimeters, and in some examples, the distal radius of curvature R2 may be in a range between four and six millimeters. For example, according to one aspect, the proximal radius of curvature R1 may be twenty-four millimeters while the distal radius of curvature R2 may be five millimeters.

The side walls 2112 may be further dimensioned to keep the needle within the central groove 2114 without blocking the optical view of the endoscope. If the side walls 2112 are too high, the optical view of the endoscope may be impeded. Conversely, if the side walls 2112 are too low, the needle may traverse the side walls 2112 and escape the central groove 2112, moving the needle out of the slicing plane. According to some embodiments, the side walls may have a height H1 in a range less than three millimeters and greater than two millimeters. For example, according to one aspect, the side walls may have a height H1 of 2.10 millimeters.

The central groove 2114 defines the path of the needle from the endoscope to the slicing plane. Because the slicing plane is not laterally aligned with a central axis defined by the body of the endoscope, the needle may deform along its path from the endoscope to the slicing plane. According to some embodiments, the central groove is dimensioned to position the needle within the slicing plane without adding undue strain to the needle. The needle guide ramp may define a length L1 and a height H2 for the needle to travel as it traverses the central groove. According to some embodiments, the length L1 may be in a range between eleven and fifteen millimeters, and in some embodiment the length L1 may be in a range between thirteen and fourteen millimeters. For example, according to one aspect, the length L1 may be 13.5 millimeters. According to some embodiments, the height H2 may be in a range between seven and ten millimeters, and in some embodiments, the height H2 may be in a range between eight and nine millimeters. For example, according to one aspect, the height H2 may be 8.4 millimeters.

According to some embodiments, the central groove 2114 may define a non-linear path between the proximal end 2118 and the distal end 2116. According to some examples, the non-linear path may follow a curve having a radius of curvature R3. If the radius of curvature R3 of the central groove 2114 is too high, the needle may deflect out of the endoscope too early. Conversely, if the radius of curvature R3 of the central groove 2114 is too low, friction from the central groove 2114 may prevent the needle from sliding through the v-shaped channel 2110. According to some examples, the groove radius of curvature R3 may be in a range between twenty to forty millimeters, and in some examples, the groove radius of curvature R3 may be in a range between thirty-three to thirty-five millimeters. For example, according to one aspect, the groove radius of curvature R3 may be thirty-five millimeters.

According to some embodiments, an edge of the needle guide ramp 2100 may be rounded by a radius of curvature R4 at the distal end 2116 to allow the needle to exit the v-shaped channel without additional resistance. Further, the edge may be oriented at an angle θ3 relative to the axis defined by the height dimension H2. According to some embodiments, the edge radius of curvature R4 may be in a range between 0.75 and 1.05 millimeters. For example, according to one aspect, the edge radius of curvature R4 may be 0.9 millimeters. According to some embodiments, the edge angle θ3 may be in a range between twenty and forty degrees, and in some embodiments, the edge angle θ3 may be in a range between twenty-five and thirty-five degrees. For example, according to one aspect, the edge angle θ3 may be thirty degrees.

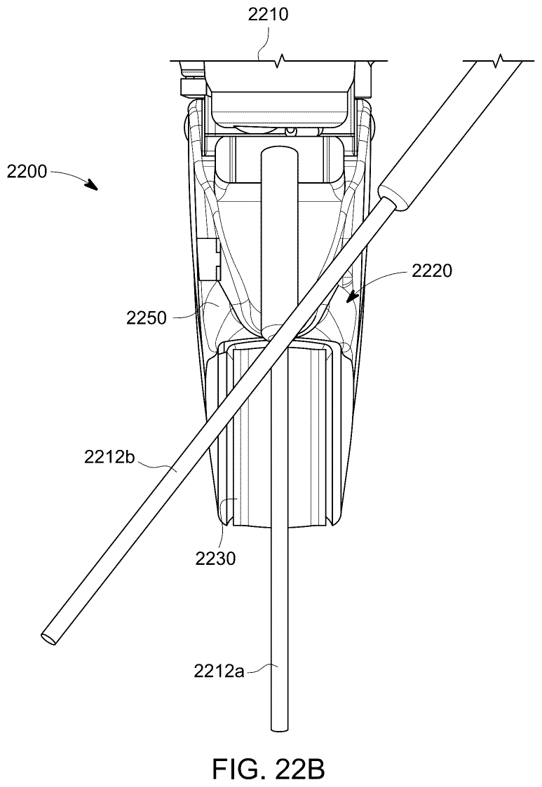

Referring now to FIGS. 22A and 22B, various views of an imaging assembly 2200 having an endoscope 2210 and an ultrasound imaging assembly 2220 comprising a needle guide ramp 2250 are depicted, according to a non-limiting embodiment of the present disclosure. The ultrasound imaging assembly 2220 may include an ultrasound imaging head 2230, an ultrasound imaging sub-assembly 2240, and a needle guide ramp 2250. The ultrasound imaging sub-assembly 2240 may comprise an abutment 2242.

The ultrasound imaging head 2230 may define the field of view in the form of a slicing plane 2252. The endoscope 2210 comprises a needle 2212a, 2212b. Although the needle 2212a, 2212b is shown as discontinuous from the endoscope 2210 in FIG. 22B, it should be appreciated that the needle 2212a, 2212b traverses through a conduit channel in the endoscope 2210 and extends from a distal end of the endoscope 2210 as shown in FIG. 22A. Upon exiting the conduit channel of the endoscope 2210, the needle may traverse the needle guide ramp 2250 to enter the slicing plane 2252. As shown in FIG. 22B, the orientation of the needle 2212a, 2212b, may affect the view within the slicing plane 2252. When the needle 2212a travels along the orientation of the slicing plane 2252, a line is visible within the slicing plane 2252. Conversely, if the needle 2212b is askew from the orientation of the slicing plane 2252, only a dot appears in the slicing plane 2252 where the needle crosses the slicing plane 2252. Proper alignment (See 2212a) generates a one-dimensional line, while improper alignment (See 2212b) generates a single coordinate across the slicing plane 2252. The needle guide ramp 2250 improves the alignment of the needle 2212a within the slicing plane 2252.

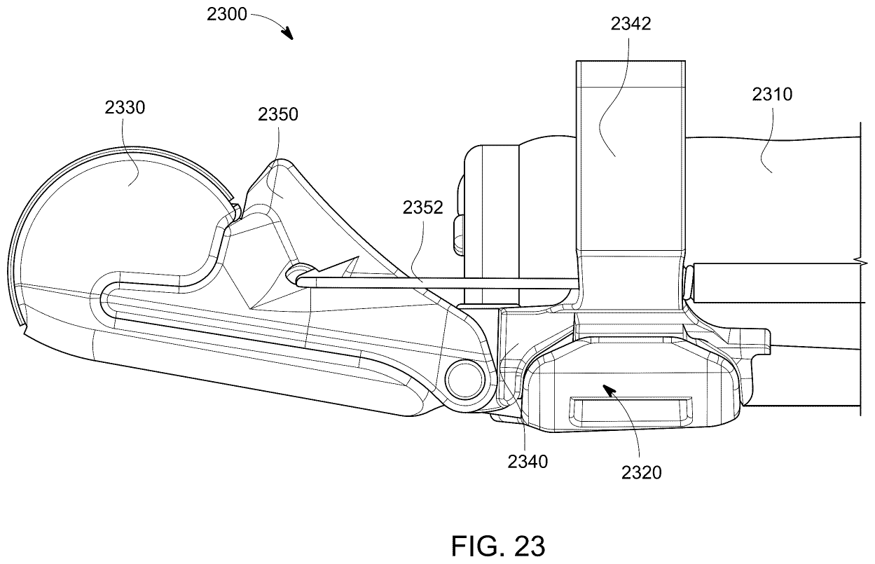

Referring now to FIG. 23, a side view of an imaging assembly 2300 having an endoscope 2310 and an ultrasound imaging assembly 2320 is depicted, according to a non-limiting embodiment of the present disclosure. The ultrasound imaging assembly 2320 may comprise an ultrasound imaging head 2330, an ultrasound imaging sub-assembly 2340, a needle guide ramp 2350, and a tension member 2352. The ultrasound imaging sub-assembly 2340 may comprise a retaining element 2342 to selectively engage the endoscope 2310.

The needle guide ramp 2350 may be configured to change direction relative to the endoscope 2310, when the ultrasound imaging assembly 2320 is attached to the endoscope 2310. The change in direction may be in response to varying user input provided via the tension member 2352. The tension member 2352 may connect to the needle guide ramp 2350 to facilitate the change in motion. According to some embodiments, the tension member 2352 may be a cable. According to some embodiments, the tension member 2352 may run along an axis parallel to the axis defined by the body of the endoscope 2310. The tension member 2352 may operate outside of the slicing plane discussed with FIGS. 22A and 22B. The tension member 2352 may be positioned such that it is outside of a field of view of the ultrasound imaging head 2330.

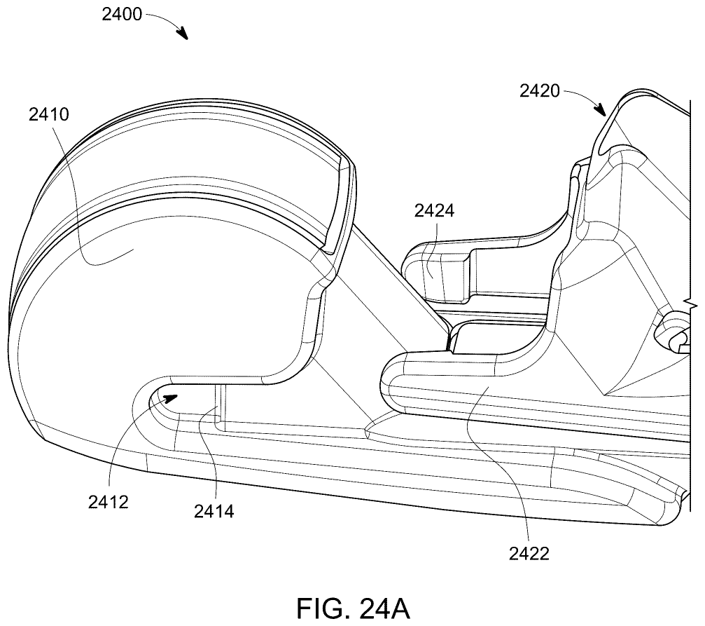

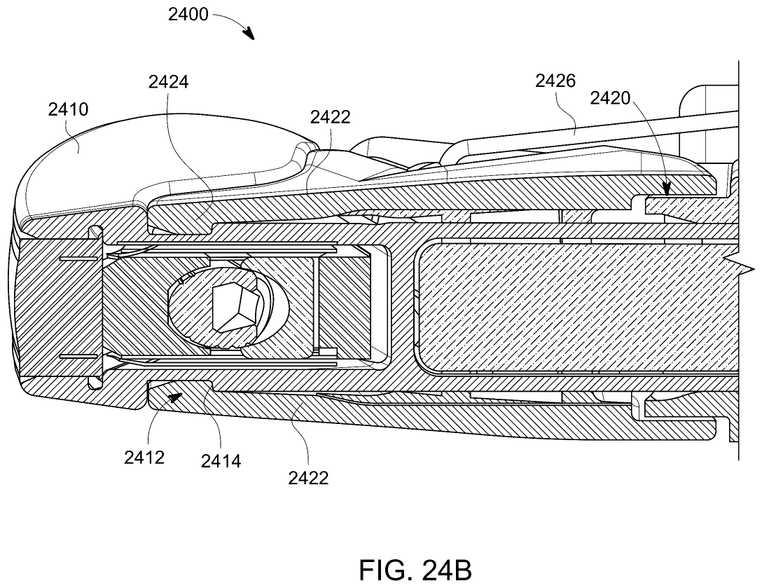

Referring now to FIGS. 24A and 24B, various views of an ultrasound imaging assembly 2400 are depicted, according to a non-limiting embodiment of the present disclosure. The ultrasound imaging assembly 2400 may comprise an ultrasound imaging head 2410 and an ultrasound imaging sub-assembly 2420. The ultrasound imaging sub-assembly 2420 may be configured to selectively engage the ultrasound imaging head 2410. FIG. 24A depicts the ultrasound imaging head 2410 and the ultrasound imaging sub-assembly 2420 prior to engagement. FIG. 24B depicts the ultrasound imaging head 2410 and the ultrasound imaging sub-assembly 2420 while selectively engaged.

According to some embodiments, the ultrasound imaging sub-assembly 2420 may comprise attachment arms 2422 to selectively engage the ultrasound imaging head 2410. According to some embodiments, the attachment arms 2422 may attach to the ultrasound imaging head 2410 via snap-fit. The attachment arms 2422 may extend outwardly from the ultrasound imaging sub-assembly 2420. According to some embodiments, the ultrasound imaging sub-assembly 2420 may comprise two attachment arms 2422. The attachment arms 2422 may be composed of a semiflexible material, allowing the attachment arms 2422 to transition between a neutral and a biased position. While in the neutral position, the attachment arms 2422 may selectively engage the ultrasound imaging head 2410. According to some embodiments, the attachment arms may run parrel to each respective attachment arm 2422 while in the neutral position. While in the biased position, the attachment arms 2422 may be deformed to either engage or disengage the ultrasound imaging head 2410. According to some embodiments, while in the biased position, the attachment arms 2422 may be closer together at the point of attachment to the sub-assembly 2420, and the attachment arms 2422 may be further apart at the point further from the sub-assembly 2420. The attachment arms 2422 may comprise an attachment tooth 2424 to engage the ultrasound imaging head 2410.

According to some embodiments, the ultrasound imaging head 2410 may define a recess 2412 configured to receive the attachment arms 2422. The recess 2412 may be sized with a mirrored geometry to the attachment arms 2422, allowing the attachment arms 2422 to slot into the recess while selectively engaged. The recess may be tapered to prevent lateral movement of the ultrasound sub-assembly while selectively engaged. According to some embodiments, the recess 2412 may be further defined by a recess wall 2414 configured to engage the tooth 2424 of the attachment arm 2422. The recess 2412 may have a depth approximately equal to a width of the attachment arm 2422, such that when the attachment arm 2422 is engaged with the recess 2412, the ultrasound imaging head 2410 and the ultrasound imaging sub-assembly 2420 sit flush without protrusions from the attachment arms 2422.

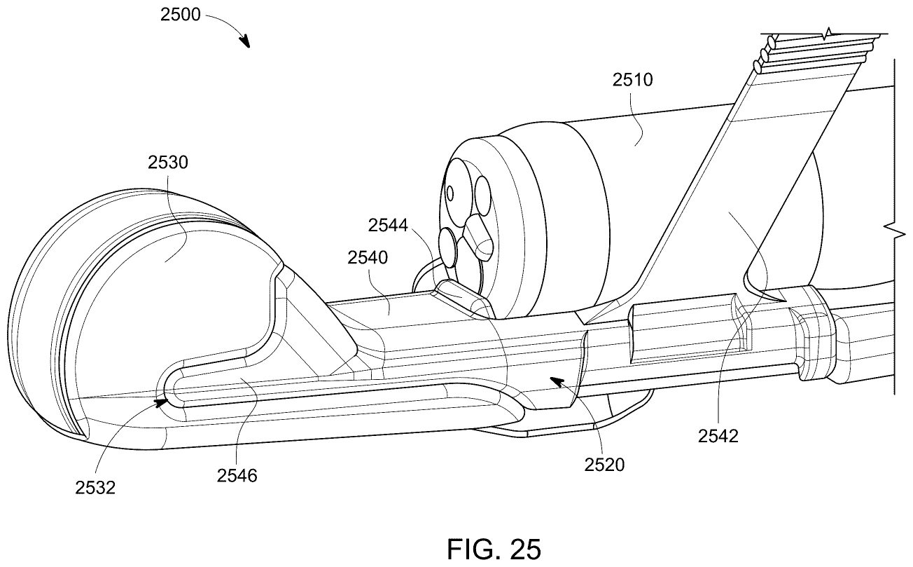

Referring now to FIG. 25, a view of an imaging assembly 2500 is depicted, according to a non-limiting embodiment of the present disclosure. The imaging assembly 2500 may comprise an endoscope 2510 and an ultrasound imaging assembly 2520. The ultrasound imaging assembly 2520 may comprise an ultrasound imaging head 2530 and an ultrasound imaging sub-assembly 2540. The ultrasound imaging sub-assembly may comprise a retention element 2542, an abutment 2544, and an attachment arm 2546.

The abutment 2544 may be positioned to prevent lateral motion of the endoscope 2510 relative to the ultrasound imaging assembly 2520 while selectively engaged. Although the abutment 2544 is shown as blocking a conduit channel of the endoscope 2510, it should be appreciated that the abutment 2544 may provide necessary clearance for a needle to traverse the conduit channel of the endoscope 2510. Such clearance may be provided by dimensioning the abutment 2544 below the conduit channel or defining an aperture in the abutment 2544 to provide clearance. The abutment 2544 may be positioned at a predetermined position relative to a distal end of the ultrasound imaging head 2530. The abutment 2544 may be positioned such that an end of the endoscope 2510 is between ten to fifteen millimeters from the distal end of the ultrasound imaging head 2530.

According to some embodiments, the abutment 2544 may be positioned in between the ultrasound imaging head 2530 and the endoscope 2510, wherein the endoscope 2510 abuts an edge of the abutment. According to some embodiments, the abutment 2544 may be configured to engage with a recess of the endoscope 2510. According to some embodiments, the abutment may engage with the recess of the endoscope 2510 to prevent lateral and rotational movement of the endoscope 2510 relative to the ultrasound imaging assembly 2520.

Various aspects of the subject matter described herein are set out in the following numbered clauses:

Clause 1: An endoscope add-on assembly adapted to be attached to a target endoscope. The endoscope add-on assembly includes an ultrasound imaging head, an ultrasound imaging sub-assembly, and a needle guide ramp. The ultrasound imaging head defines an imaging slice plane. The ultrasound imaging sub-assembly is configured to selectively engage the ultrasound imaging head. The ultrasound imaging sub-assembly includes a retaining element configured to secure the ultrasound imaging sub-assembly to the target endoscope. The needle guide ramp is configured to direct a motion of a needle of the target endoscope. The needle guide ramp is configured to align the needle of the target endoscope within the imaging slice plane when the endoscope add-on assembly is attached to the target endoscope.

Clause 2: The endoscope add-on assembly according to clause 1, wherein the needle guide ramp defines a v-shaped channel configured to align the needle of the target endoscope within the imaging slice plane.

Clause 3: The endoscope add-on assembly according to clause 2, wherein the v-shaped channel includes side walls and defines a central groove.

Clause 4: The endoscope add-on assembly according to clause 3, wherein the side walls are sized such that the needle is deflected along the central groove without blocking a view of the slicing plane from the endoscope.

Clause 5: The endoscope add-on assembly according to clause 3, wherein the side walls taper such that the side walls define a narrower opening at a distal end.

Clause 6: The endoscope add-on assembly according to clause 5, wherein the v-shaped channel defines a proximal angle of the side walls at a proximal end of the needle guide ramp, wherein the v-shaped channel defines a distal angle of the side walls at the distal end of the needle guide ramp, and wherein the proximal angle is greater than the distal angle.

Clause 7: The endoscope add-on assembly according to clause 2, wherein the v-shaped channel defines a radius of curvature between a proximal end and the distal end of the needle guide ramp in a range of twenty to forty millimeters.

Clause 8: The endoscope add-on assembly according to clause 1, wherein the needle guide ramp is between eleven to fifteen millimeters in length.

Clause 9: The endoscope add-on assembly according to clause 1, wherein the needle guide ramp is configured to change direction relative to the target endoscope, when the endoscope add-on assembly is attached to the target endoscope, in response to a varying user input provided via a tension member including a compressive strength.

Clause 10: The endoscope add-on assembly according to clause 9, wherein the tension member is a cable.

Clause 11: An endoscope add-on assembly adapted to be attached to a target endoscope. The endoscope add-on assembly includes an ultrasound imaging head and an ultrasound imaging sub-assembly. The ultrasound imaging sub-assembly is configured to selectively engage the ultrasound imaging head. The ultrasound imaging sub-assembly includes a retaining element and an abutment. The retaining element is configured to secure the ultrasound imaging sub-assembly to the target endoscope. The abutment is configured to align the ultrasound imaging sub-assembly in a first predetermined position relative to the target endoscope when secured.

Clause 12: The endoscope add-on assembly according to clause 11, wherein the abutment is positioned at a second predetermined position from a distal end of the ultrasound imaging head.

Clause 13: The endoscope add-on assembly according to clause 12, wherein the abutment is positioned at the second predetermined position such that the endoscope is between ten to fifteen millimeters from the distal end of the ultrasound imaging head while the endoscope add-on assembly is selectively engaged with the endoscope.

Clause 14: The endoscope add-on assembly according to clause 11, wherein the abutment has a first geometry configured to selectively engage a recess defining an inverse geometry of the endoscope, such that the abutment prevents lateral and rotational movement of the endoscope relative to the endoscope add-on assembly while the endoscope add-on assembly is selectively engaged with the endoscope.

Clause 15: An endoscope add-on assembly adapted to be attached to a target endoscope. The endoscope add-on assembly includes an ultrasound imaging head and an ultrasound imaging sub-assembly. The ultrasound imaging head defines a recess. The ultrasound imaging sub-assembly includes an attachment arm and a retaining element. The attachment arm has a geometry compatible to the recess. The attachment arm is configured to selectively engage the ultrasound imaging head. The retaining element is configured to secure the ultrasound imaging sub-assembly to the target endoscope.

Clause 16: The endoscope add-on assembly according to clause 15, wherein the attachment arm selectively engages with the ultrasound imaging head via a snap fit.

Clause 17: The add-on assembly according to clause 16, wherein the attachment arm is composed of a semi-flexible material to selectively engage the ultrasound imaging head.

Clause 18: The endoscope add-on assembly according to clause 17, wherein the attachment arm is configured to transition between a neutral position and a biased position. The attachment arm selectively engages the ultrasound imaging head while in the neutral position. The attachment arm disengages the ultrasound imaging head while in the biased position.

Clause 19: The endoscope add-on assembly according to clause 16, wherein the ultrasound imaging head defines a wall of the recess. The attachment arm includes an attachment tooth configured to selectively engage with the wall of the recess.

Clause 20: The endoscope add-on assembly according to clause 19, wherein the attachment tooth and the wall of the recess are at an angle relative to the attachment arm such that the endoscope add-on assembly remains selectively engaged with the endoscope during operation.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. An endoscope add-on assembly adapted to be attached to a target endoscope, the endoscope add-on assembly including:
an ultrasound imaging head defining an imaging slice plane;
an ultrasound imaging sub-assembly configured to selectively engage the ultrasound imaging head, wherein said ultrasound imaging sub-assembly comprises a retaining element configured to secure the ultrasound imaging sub-assembly to the target endoscope; and
a needle guide ramp configured to direct a motion of a needle of the target endoscope, wherein the needle guide ramp is configured to align the needle of the target endoscope within the imaging slice plane when the endoscope add-on assembly is attached to the target endoscope.

2. The endoscope add-on assembly of claim 1, wherein the needle guide ramp defines a v-shaped channel configured to align the needle of the target endoscope within the imaging slice plane.

3. The endoscope add-on assembly of claim 2, wherein the v-shaped channel comprises side walls and defines a central groove.

4. The endoscope add-on assembly of claim 3, wherein the side walls are sized such that the needle is deflected along the central groove without blocking a view of the slicing plane from the endoscope.

5. The endoscope add-on assembly of claim 3, wherein the side walls taper such that the side walls define a narrower opening at a distal end.

6. The endoscope add-on assembly of claim 5, wherein the v-shaped channel defines a proximal angle of the side walls at a proximal end of the needle guide ramp, wherein the v-shaped channel defines a distal angle of the side walls at the distal end of the needle guide ramp, and wherein the proximal angle is greater than the distal angle.

7. The endoscope add-on assembly of claim 2, wherein the v-shaped channel defines a radius of curvature between a proximal end and the distal end of the needle guide ramp in a range of twenty to forty millimeters.

8. The endoscope add-on assembly of claim 1, wherein the needle guide ramp is between eleven to fifteen millimeters in length.

9. The endoscope add-on assembly of claim 1, wherein the needle guide ramp is configured to change direction relative to the target endoscope, when the endoscope add-on assembly is attached to the target endoscope, in response to a varying user input provided via a tension member comprising a compressive strength.

10. The endoscope add-on assembly of claim 9, wherein the tension member is a cable.

11. An endoscope add-on assembly adapted to be attached to a target endoscope, the endoscope add-on assembly including:
an ultrasound imaging head; and
an ultrasound imaging sub-assembly configured to selectively engage the ultrasound imaging head, wherein said ultrasound imaging sub-assembly comprises:
a retaining element configured to secure the ultrasound imaging sub-assembly to the target endoscope; and
an abutment configured to align the ultrasound imaging sub-assembly in a first predetermined position relative to the target endoscope when secured.

12. The endoscope add-on assembly of claim 11, wherein the abutment is positioned at a second predetermined position from a distal end of the ultrasound imaging head.

13. The endoscope add-on assembly of claim 12, wherein the abutment is positioned at the second predetermined position such that the endoscope is between ten to fifteen millimeters from the distal end of the ultrasound imaging head while the endoscope add-on assembly is selectively engaged with the endoscope.

14. The endoscope add-on assembly of claim 11, wherein the abutment has a first geometry configured to selectively engage a recess defining an inverse geometry of the endoscope, such that the abutment prevents lateral and rotational movement of the endoscope relative to the endoscope add-on assembly while the endoscope add-on assembly is selectively engaged with the endoscope.

15. An endoscope add-on assembly adapted to be attached to a target endoscope, the endoscope add-on assembly including:
an ultrasound imaging head defining a recess; and
an ultrasound imaging sub-assembly comprising:
an attachment arm having a geometry compatible to the recess, the attachment arm configured to selectively engage the ultrasound imaging head; and
a retaining element configured to secure the ultrasound imaging sub-assembly to the target endoscope.

16. The endoscope add-on assembly of claim 15, wherein the attachment arm selectively engages with the ultrasound imaging head via a snap fit.

17. The add-on assembly of claim 16, wherein the attachment arm is composed of a semi-flexible material to selectively engage the ultrasound imaging head.

18. The endoscope add-on assembly of claim 17, wherein the attachment arm is configured to transition between a neutral position and a biased position, wherein the attachment arm selectively engages the ultrasound imaging head while in the neutral position, and wherein the attachment arm disengages the ultrasound imaging head while in the biased position.

19. The endoscope add-on assembly of claim 16, wherein the ultrasound imaging head defines a wall of the recess, and wherein the attachment arm comprises an attachment tooth configured to selectively engage with the wall of the recess.

20. The endoscope add-on assembly of claim 19, wherein the attachment tooth and the wall of the recess are at an angle relative to the attachment arm such that the endoscope add-on assembly remains selectively engaged with the endoscope during operation.
